(19) United States
(12) Patent Application Publication
(10) Pub. No.: US 2021/0069093 A1
(43) Pub. Date: Mar. 11, 2021

(54) MAKEUP COMPOSITIONS FOR TREATING SKIN DEFECTS

(71) Applicant: Joel STUDIN, Roslyn, NY (US)

(72) Inventor: Joel STUDIN, Roslyn, NY (US)

(21) Appl. No.: 16/953,811

(22) Filed: Nov. 20, 2020

Related U.S. Application Data
(63) Continuation of application No. 16/208,087, filed on Dec. 3, 2018.

(51) Int. Cl. A61K 8/891; A61K 8/63; A61K 8/29; A61Q 1/02

(52) U.S. Cl. CPC A61K 8/891; A61K 8/63; A61Q 17/04; A61Q 1/02; A61K 2800/621; A61K 8/29

(57) ABSTRACT

The present disclosure is related to a method of treating and concealing a skin defect in a subject. The method comprises applying a makeup composition to the skin defect, wherein the makeup composition comprises silicone and a coloring agent, wherein the makeup composition simultaneously conceals and treats said skin defect. Makeup compositions and kits for treating and concealing skin defects are also disclosed.

### FIELD OF THE INVENTION

The present invention relates to methods for treating and concealing a skin defect in a subject in need thereof via the application of silicone-based makeup compositions. The present invention also relates to makeup compositions and kits for treatment.

### BACKGROUND

Makeup compositions are used to enhance or emphasize certain facial features, such as the eyes or lips. Another important use is to camouflage the appearance of facial or other bodily features that are less than perfect. Few consumers are blessed with flawless skin, and concealment of skin flaws is often desired. For example, adults or teens who suffered from acne may desire to apply a makeup composition to conceal acne scars and pimples.

Consumers do not have makeup compositions engineered to treat skin defects, especially scars. In some situations, some makeup compositions delay or even worsen the healing process of certain skin defects. Conversely, consumers often use products that help heal skin defects but these do not conceal the defect during the healing process. For example, U.S. Pat. No. 5,759,560 discloses silicone thermoplastic sheeting suitable for use in the treatment of dermatologic scars; U.S. Pat. No. 6,284,941 discloses a bandage comprising silicone gels for the treatment of dermal scars, keloids, wounds or abrasions; and U.S. Pat. No. 6,159,494 discloses a tape containing a gel from Bulbine frutescens to treat post-operative scars. None of these products can simultaneously conceal and heal scars.

The shrinkage of hypertrophic scars can be increased by applying silicone-gel plates or sheets to the scars. A number of products are available commercially for this purpose, for instance, Dow Corning Silastic Sheeting, Cica-Care (Smith & Nephew), Epi-Derm (Biodermis), and Nagosil (Nagor), among others. These products have the form of molded silicone-gel sheets having a thickness of 2-4 millimeters. In treating hypertrophic scars, these sheets are placed over the scars and are worn for a relatively long period of time, often from 3-12 months, until the scars either have decreased or are removed. These products are large, very unsightly and unwieldy. Examples of patents which disclose such silicone-gel sheets include U.S. Pat. Nos. 5,759,560; 5,891,076; 5,895,656 and 5,919,476. None of these products provide an aesthetic effect to the skin as makeup compositions do.

As the healing process for many skin defects, such as severe skin scarring can be lengthy, and these defects can be upsetting, there is a need for makeup compositions that incorporate effective scar treatment technology to simultaneously conceal and heal skin defects.

### BRIEF SUMMARY OF THE INVENTION

According to various aspects and embodiments of this disclosure, the present invention is directed to makeup compositions, kits, and methods for treating a skin defect in a subject in need thereof via the application of high silicone content makeup-scar treatment compositions.

In one embodiment, the method of treating and concealing a skin defect in a subject comprises applying a makeup composition to the skin defect, wherein the makeup composition comprises at least 5%, 10%, or 20% by weight silicone and a coloring agent, wherein the makeup composition simultaneously conceals and treats said skin defect. In another embodiment, the makeup composition is silky smooth, non-greasy, and odorless. In another embodiment, the makeup composition is not comprised of immiscible layers.

In another embodiment, the makeup composition further comprises a scar melting agent. In one embodiment, the scar melting agent is selected from the group consisting of one or more steroid hormones, collagenases, proteases, oligopolysaccharides, and biologics, or combinations thereof. In one embodiment, the scar melting agent is a steroid hormone. In a preferred embodiment, the steroid hormone is cortisone.

In another embodiment, the skin defect is an existing scar or a healing wound from C-section, plastic surgery, heart surgery, pediatric cleft lip, orthopedic surgery, burn, cut, abrasion, or other traumatic injury.

In another embodiment, the scar is selected from a keloid scar, a hypertrophic scar, a contracture scar, an acne scar, a chicken pox scar, skin cancer scar, a dog bite scar, an accident scar, or a surgical scar.

In another embodiment, the scar is a surgical scar following suture removal about 7 to about 14 days after surgery.

In another embodiment, the makeup composition further provides UV protection.

In another embodiment, the scar is substantially healed after the treatment. In another embodiment, the treatment results in substantial reduction in the amount and visibility of scar tissue. In one embodiment, the size of the scar is reduced at least 10%, at least 20%, at least 30%, at least 40%, or at least 50% after the treatment.

In another embodiment, the treatment further provides a calming or soothing effect to the skin.

In another embodiment, the makeup composition contains a nacreous pigment. In one embodiment, the nacreous pigment is titanium mica with iron oxides.

In another embodiment, the silicone is selected from liquid silicone, oil silicone, wax silicone, resin silicone, or mixtures thereof.

In another embodiment, the silicone is selected from dimethicone, methicone, trimethicone, cyclomethicone, amodimethicone, trimethylsilylamodimethicone, cyclotetrasiloxane, cyclopentasiloxane, cyclehexasiloxane, polydimethylsiloxane, polymethylsilsequioxane, and dimethiconol, and mixtures thereof.

In another embodiment, the volume of the makeup composition applied to the skin defect is about 0.1 to about 10 mL.

In another embodiment, the viscosity of the silicone is about 1000 cP to about 1,000,000 cP.

In another embodiment, the makeup composition is maintained on the skin for at least 1-8 hours daily.

In another embodiment, the makeup composition is applied 1-5 times daily for about 14 days to about 120 days.

In another embodiment, the weight of silicone ranges from about 5% to about 90% with respect to the total weight of the makeup composition. In one embodiment, the weight of silicone is at least 5%, at least 10%, at least 15%, at least 20%, at least 30%, at least 40%, at least 50%, at least 60%, at least 70%, at least 80%, or at least 90% with respect to the total weight of the makeup composition.

In another embodiment, the subject is a human.

In another embodiment, the present disclosure provides a makeup composition comprising at least 80% by weight of silicone and a coloring agent, wherein the composition, when applied to a scar on the skin of a subject, treats said scar on the skin of the subject while simultaneously camouflaging the scar and providing UV protection. In one embodiment, the makeup composition is not comprised of immiscible layers.

In another embodiment, the silicone comprises polydimethylsiloxane.

In another embodiment, a scar treatment kit is provided, comprising a) the makeup composition described above; b) at least one container which contains the makeup composition; and c) at least one applicator.

In another embodiment, the applicator is a brush, puff, or sponge.

### DETAILED DESCRIPTION OF THE INVENTION

### Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

### Definition of General Terms

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

The term “and/or” when used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “substantial” as used herein is used in its ordinary sense, including, without limitation, an amount greater than 50 percent.

### Definition of Specific Terms

The term “skin defect” as used herein is defined as any abnormality or flaw related to the skin or epidermis. Deficiencies may include skin lesions, scars, hyperpigmentation, hypopigmention, atrophy, burn, trauma, or wound.

The term “makeup composition” as used herein is defined as a product comprising a coloring agent that allows the deposition of a color onto a keratinous material such as the skin or the integuments of a human, by applying onto the keratinous material products such as lipsticks, makeup powders, makeup liquid, mineral makeup, eyeliners, foundations, self-tanning agents or semi-permanent makeup products (tattoos). In one embodiment, the color being deposited onto the keratinous material matches that of the keratinous material.

The term “immiscible” as used herein is defined as incapable of combining to form a homogeneous mixture.

The term “steroid hormone” refers to a hormone having a steroid skeleton. Examples of steroid hormones may include, but are not limited to, cortisone, clobetasol propionate, flurandrenolide, betamethasone dipropionate, diflorasone diacetate, desoximetasone, halobetasol propionate, fluocinonide, mometasone furoate, mometasone, halcinonide, desoximetasone, fluticasone propionate, triamcinolone acetonide, hydrocortisone valerate, fluocinolone acetonide, prednicarbate, desonide, hydrocortisone, fluocinolone acetonide, hydrocortisone valerate, alclometasone dipropionate, and pharmaceutically acceptable salts thereof.

The term “collagenase” as used herein refers to proteolytic enzymes which are capable of degrading native collagen.

The term “protease” as used herein refers to an enzyme that catalyzes the cleavage of peptide bonds, e.g., in proteins, polypeptides, oligopeptides, and peptides (collectively “peptides”). Exemplary proteases include, but are not limited to, serine proteases, cysteine proteases, threonine proteases, aspartic proteases, glutamic proteases, metalloproteases, asparagine peptide lyases, and the like.

The term “oligopolysaccharide” as used herein refers to a molecule composed of more than a single saccharide unit where these units are connected through glycosidic linkages. As used within this application, the term oligopolysaccharide would also include high molecular weight oligomers known as polysaccharides.

The term “biologics” as used herein refers to biological drugs that include a wide range of products such as vaccines, blood and blood components, allergenics, somatic cells, gene therapy, tissues, and proteins. Biologics can be composed of sugars, proteins, or nucleic acids or complex combinations of these substances, or may be living entities such as cells and tissues. Biologics can be isolated from a variety of sources-human, animal, or microorganism.

The term “keloid scar” as used herein is defined as a result of an overgrowth of granulation tissue at the site of a healed skin injury. It can be an overgrown scar, or an overly aggressive healing response to a wound.

The term “hypertrophic scar” as used herein is defined as a non-proliferative lump that is a red raised lump on the skin.

The term “contracture scar” as used herein refers to the result of the contractile process occurring in a healed scar, which often results in an undesirable, fixed, rigid scar that can cause functional or cosmetic deformity. Contracture scars are reviewed by Ross Rudolph, Jerry Vande Berg and H. Paul Ehrlich in Wound Healing: Biochemical and Clinical Aspects, I. K. Cohen, R. F. Diegelmann and W. J. Lindblad Eds, W. B. Saunders Company, pages 96-114(1992).

The term “acne scar” as used herein refers to scars formed in acne-affected skin. Acne is often accompanied by scarring (scar formation). The term “acne” includes mild acne, moderate acne, severe acne and very severe acne. Mild acne refers to the presence of few skin lesions, such as few to several papules/pustules and nodules; moderate acne refers to the presence of several to many papules/pustules, few to several nodules and numerous and extensive comedones; severe acne refers to numerous and/or extensive papules/pustules, many persistent or recurrent nodules large and very extensive comedones, ongoing scarring, persistent purulent and/or serosanguinous drainage from lesions, presence of sinus tracts; and very severe acne includes conditions termed acne conglobata, acne fulminans and acne inversa (follicular occlusion triad).

The term “surgical scar” as used herein refers to scars formed post surgical procedures.

The term “UV protection” as used herein refers to protection against the harmful effects of ultraviolet radiation.

The terms “color agent” and “coloring agent” as used herein refer to any pigment or dye or mixture of pigments and/or dyes suitable for providing sufficient color to a makeup composition. In one embodiment, the coloring agent is used to match the color of the makeup composition to the color of the keratinous material on which the composition is being deposited.

The term “silicone” as used herein refers to organosilicon polymers or oligomers with a linear or cyclic, branched or crosslinked structure, of variable molecular weight, obtained by polymerization and/or polycondensation of suitably functionalized silanes and essentially composed of a repetition of main units in which the silicon atoms are connected to one another via oxygen atoms (siloxane bond ≡Si—O—Si≡), optionally substituted hydrocarbonaceous radicals being directly bonded via carbon atom to the said silicon atoms. The most common hydrocarbonaceous radicals are alkyl radicals, in particular (C₁-C₃₀) alkyl radicals and especially methyl radicals, fluoroalkyl radicals or aryl radicals, in particular the phenyl radical.

The term “liquid silicone” as used herein refers to a silicone compound which is liquid at ambient temperature (25° C.) and atmospheric pressure (760 mm Hg).

The term “silicone oil” as used herein refers broadly to an oil comprising at least one silicon atom, and in particular comprising Si—O groups.

The term “silicone wax” as used herein refers to silicone products which are semi-solids or solids at room temperature. The examples of silicone wax include a polydimethylsiloxane having a substituted long chain alkyl (such as C₁₆ to C₃₀), such as stearyl dimethicone, bis-stearyl dimethicone, behenyl dimethicone, or behenoxy dimethicone.

The term “silicone resin” as used herein refers to a series of silicone polymers which include at least two silicone backbones that are joined by a crosslinking group.

The term “viscosity” as used herein refers to the measure of fluid friction. A highly viscous material is one that possesses a great deal of internal friction, and will not pour or spread as easily as material of lesser viscosity.

### Methods of Treating a Skin Defect

The present disclosure describes a method for treating and concealing a skin defect in a subject. The method comprises applying a makeup composition to the skin defect, wherein the makeup composition comprises at least 5%, 10% or 20% by weight silicone and a coloring agent, wherein the makeup composition simultaneously conceals and treats said skin defect.

In another embodiment, the weight of silicone ranges from about 5% to about 90% with respect to the total weight of the makeup composition. In one embodiment, with respect to the total weight of the makeup composition, the weight of silicone ranges from about 5% to about 90%, about 5% to about 85%, about 5% to about 80%, about 5% to about 75%, about 5% to about 70%, about 5% to about 65%, about 5% to about 60%, about 5% to about 55%, about 5% to about 50%, about 5% to about 45%, about 5% to about 40%, about 5% to about 35%, about 5% to about 30%, about 5% to about 25%, about 5% to about 20%, about 5% to about 15%, about 5% to about 10%, about 10% to about 90%, about 15% to about 90%, about 20% to about 90%, about 25% to about 90%, about 30% to about 90%, about 35% to about 90%, about 40% to about 90%, about 45% to about 90%, about 50% to about 90%, about 55% to about 90%, about 60% to about 90%, about 65% to about 90%, about 70% to about 90%, about 75% to about 90%, about 80% to about 90%, and about 85% to about 90%. In one embodiment, the weight of silicone is at least 5%, at least 10%, at least 15%, at least 20%, at least 30%, at least 40%, at least 50%, at least 60%, at least 70%, at least 80%, or at least 90% with respect to the total weight of the makeup composition.

In one embodiment, the composition is not comprised of immiscible layers.

In another embodiment, the makeup composition further comprises a scar melting agent. In one embodiment, the scar melting agent is selected from the group consisting of one or more steroid hormones, collagenases, proteases, oligopolysaccharides, and biologics, or combinations thereof. In one embodiment, the scar melting agent is a steroid hormone. In a preferred embodiment, the steroid hormone is cortisone.

In one embodiment in connection with the method, the skin defect is any abnormality or flaw related to the skin or epidermis. Skin defects may include, but are not limited to, skin lesions, scars, hyperpigmentation, hypopigmention, atrophy, burn, trauma, or wound. In a preferred embodiment, the skin defect is a scar.

In one embodiment in connection with the method, the silicone refers to organosilicon polymers or oligomers with a linear or cyclic, branched or crosslinked structure, of variable molecular weight, obtained by polymerization and/or polycondensation of suitably functionalized silanes and essentially composed of a repetition of main units in which the silicon atoms are connected to one another via oxygen atoms (siloxane bond ≡Si—O—Si≡), optionally substituted hydrocarbonaceous radicals being directly bonded via carbon atom to the said silicon atoms. The most common hydrocarbonaceous radicals are alkyl radicals, in particular (C₁-C₃₀) alkyl radicals and especially methyl radicals, fluoroalkyl radicals or aryl radicals, in particular the phenyl radical.

A variety of silicones can be used in the composition. The silicone can be chosen from silicone liquid, silicone oils, which may or may not be volatile, silicone waxes, silicone gums, silicone resins, silicone surfactants and/or their mixtures.

In one embodiment, silicone oil is a compound corresponding to the following general formula (I):

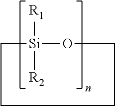

wherein R₁ and R₂ are an independent C₁₋₆ alkyl;
n is in integer from 3-100.

In one embodiment, silicone oil is linear polydimethylsiloxanes (PDMSs) corresponding to the following general formula (II):

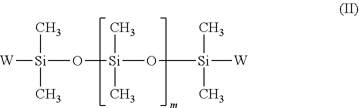

wherein

W is —CH₃ or —OH, and

m is an integer from 0 to 2000.

In one embodiment in connection with the method, the weight of silicone oil in the makeup composition can range from about 1% to about 99%, about 1% to about 90%, about 1% to about 80%, about 1% to about 70%, about 1% to about 60%, about 1% to about 50%, about 1% to about 40%, about 1% to about 30%, about 1% to about 20%, about 1% to about 10%, about 1% to about 5%, about 5% to about 99%, about 5% to about 90%, about 5% to about 80%, about 5% to about 70%, about 5% to about 60%, about 5% to about 50%, about 5% to about 40%, about 5% to about 30%, about 5% to about 20%, about 5% to about 10%, about 10% to about 99%, about 10% to about 90%, about 10% to about 80%, about 10% to about 70%, about 10% to about 60%, about 10% to about 50%, about 10% to about 40%, about 10% to about 30%, about 10% to about 20%, about 15% to about 99%, about 15% to about 90%, about 15% to about 80%, about 15% to about 70%, about 15% to about 60%, about 15% to about 50%, about 15% to about 40%, about 15% to about 30%, about 15% to about 20%, about 20% to about 99%, about 20% to about 90%, about 20% to about 80%, about 20% to about 70%, about 20% to about 60%, about 20% to about 50%, about 20% to about 40%, about 20% to about 30%, about 25% to about 99%, about 25% to about 90%, about 25% to about 80%, about 25% to about 70%, about 25% to about 60%, about 25% to about 50%, about 25% to about 40%, about 25% to about 30%, about 30% to about 99%, about 30% to about 90%, about 30% to about 80%, about 30% to about 70%, about 30% to about 60%, about 30% to about 50%, about 30% to about 40%, about 35% to about 99%, about 35% to about 90%, about 35% to about 80%, about 35% to about 70%, about 35% to about 60%, about 35% to about 50%, about 35% to about 40%, about 40% to about 99%, about 40% to about 90%, about 40% to about 80%, about 40% to about 70%, about 40% to about 60%, about 40% to about 50%, about 45% to about 99%, about 45% to about 90%, about 45% to about 80%, about 45% to about 70%, about 45% to about 60%, about 45% to about 50%, about 50% to about 99%, about 50% to about 90%, about 50% to about 80%, about 50% to about 70%, about 50% to about 60%, about 55% to about 99%, about 55% to about 90%, about 55% to about 80%, about 55% to about 70%, about 55% to about 60%, about 60% to about 99%, about 60% to about 90%, about 60% to about 80%, about 60% to about 70%, about 65% to about 99%, about 65% to about 90%, about 65% to about 80%, about 65% to about 70%, about 70% to about 99%, about 70% to about 90%, about 70% to about 80%, about 75% to about 99%, about 75% to about 90%, about 75% to about 80%, about 80% to about 99%, about 80% to about 90%, about 85% to about 99%, about 85% to about 90%, about 90% to about 99%, about 90% to about 95%, or about 95% to about 99%. In another embodiment, the weight of silicone oil in the makeup composition is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99%.

In one embodiment in connection with the method, the silicone wax is a silicone product which is a semi-solid or solid at room temperature. In another embodiment, the silicone wax has a melting point ranging from about 25 to about 135° C.

In one embodiment in connection with the method, the silicone wax is a compound corresponding to the following general formula (III):

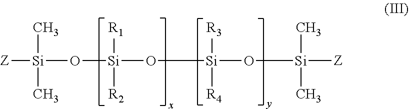

wherein

R₁, R₂, R₃, and R₄ are independently selected from C₁-C₃₀ alkyl, C₁-C₃₀ alkoxy, and aryl, wherein each group is optionally substituted by an ester group;

Z is selected from C₁-C₃₀ alkyl, C₁-C₃₀ alkoxy, C₁-C₃₀ ester, and aryl; and

x and y are independently an integer from 0 to 100.

In one embodiment in connection with the method, the weight of silicone wax in the makeup composition can range from about 1% to about 99%, about 1% to about 90%, about 1% to about 80%, about 1% to about 70%, about 1% to about 60%, about 1% to about 50%, about 1% to about 40%, about 1% to about 30%, about 1% to about 20%, about 1% to about 10%, about 1% to about 5%, about 5% to about 99%, about 5% to about 90%, about 5% to about 80%, about 5% to about 70%, about 5% to about 60%, about 5% to about 50%, about 5% to about 40%, about 5% to about 30%, about 5% to about 20%, about 5% to about 10%, about 10% to about 99%, about 10% to about 90%, about 10% to about 80%, about 10% to about 70%, about 10% to about 60%, about 10% to about 50%, about 10% to about 40%, about 10% to about 30%, about 10% to about 20%, about 15% to about 99%, about 15% to about 90%, about 15% to about 80%, about 15% to about 70%, about 15% to about 60%, about 15% to about 50%, about 15% to about 40%, about 15% to about 30%, about 15% to about 20%, about 20% to about 99%, about 20% to about 90%, about 20% to about 80%, about 20% to about 70%, about 20% to about 60%, about 20% to about 50%, about 20% to about 40%, about 20% to about 30%, about 25% to about 99%, about 25% to about 90%, about 25% to about 80%, about 25% to about 70%, about 25% to about 60%, about 25% to about 50%, about 25% to about 40%, about 25% to about 30%, about 30% to about 99%, about 30% to about 90%, about 30% to about 80%, about 30% to about 70%, about 30% to about 60%, about 30% to about 50%, about 30% to about 40%, about 35% to about 99%, about 35% to about 90%, about 35% to about 80%, about 35% to about 70%, about 35% to about 60%, about 35% to about 50%, about 35% to about 40%, about 40% to about 99%, about 40% to about 90%, about 40% to about 80%, about 40% to about 70%, about 40% to about 60%, about 40% to about 50%, about 45% to about 99%, about 45% to about 90%, about 45% to about 80%, about 45% to about 70%, about 45% to about 60%, about 45% to about 50%, about 50% to about 99%, about 50% to about 90%, about 50% to about 80%, about 50% to about 70%, about 50% to about 60%, about 55% to about 99%, about 55% to about 90%, about 55% to about 80%, about 55% to about 70%, about 55% to about 60%, about 60% to about 99%, about 60% to about 90%, about 60% to about 80%, about 60% to about 70%, about 65% to about 99%, about 65% to about 90%, about 65% to about 80%, about 65% to about 70%, about 70% to about 99%, about 70% to about 90%, about 70% to about 80%, about 75% to about 99%, about 75% to about 90%, about 75% to about 80%, about 80% to about 99%, about 80% to about 90%, about 85% to about 990%, about 85% to about 90%, about 90% to about 99%, about 90% to about 95%, or about 95% to about 99%. In another embodiment, the weight of silicone wax in the makeup composition is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99%.

In one embodiment in connection with the method, the silicone gum is a compound with a viscosity at 25° C., ranging from 500,000 cSt to 5×10⁷ cSt. In another embodiment, the silicone gum does not exhibit a melting point.

In another embodiment in connection with the method, the silicone gum is a compound corresponding to the following general formula (IV):

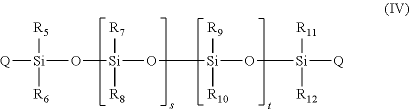

wherein
R₅, R₆, R₇, R₈, R₉, R₁₀, R₁₁, and R₁₂ are independently an C₁-C₆ alkyl;
Q is selected from C₁-C₆ alkyl, —OH, and C₂₋₁₀alkenyl;
s and t are independently an integer from 1-5000.

In one embodiment in connection with the method, the weight of silicone gum in the makeup composition can range from about 1% to about 99%, about 1% to about 90%, about 1% to about 80%, about 1% to about 70%, about 1% to about 60%, about 1% to about 50%, about 1% to about 40%, about 1% to about 30%, about 1% to about 20%, about 1% to about 10%, about 1% to about 5%, about 5% to about 99%, about 5% to about 90%, about 5% to about 80%, about 5% to about 70%, about 5% to about 60%, about 5% to about 50%, about 5% to about 40%, about 5% to about 30%, about 5% to about 20%, about 5% to about 10%, about 10% to about 99%, about 10% to about 90%, about 10% to about 80%, about 10% to about 70%, about 10% to about 60%, about 10% to about 50%, about 10% to about 40%, about 10% to about 30%, about 10% to about 20%, about 15% to about 99%, about 15% to about 90%, about 15% to about 80%, about 15% to about 70%, about 15% to about 60%, about 15% to about 50%, about 15% to about 40%, about 15% to about 30%, about 15% to about 20%, about 20% to about 99%, about 20% to about 90%, about 20% to about 80%, about 20% to about 70%, about 20% to about 60%, about 20% to about 50%, about 20% to about 40%, about 20% to about 30%, about 25% to about 99%, about 25% to about 90%, about 25% to about 80%, about 25% to about 70%, about 25% to about 60%, about 25% to about 50%, about 25% to about 40%, about 25% to about 30%, about 30% to about 99%, about 30% to about 90%, about 30% to about 80%, about 30% to about 70%, about 30% to about 60%, about 30% to about 50%, about 30% to about 40%, about 35% to about 99%, about 35% to about 90%, about 35% to about 80%, about 35% to about 70%, about 35% to about 60%, about 35% to about 50%, about 35% to about 40%, about 40% to about 99%, about 40% to about 90%, about 40% to about 80%, about 40% to about 70%, about 40% to about 60%, about 40% to about 50%, about 45% to about 99%, about 45% to about 90%, about 45% to about 80%, about 45% to about 70%, about 45% to about 60%, about 45% to about 50%, about 50% to about 99%, about 50% to about 90%, about 50% to about 80%, about 50% to about 70%, about 50% to about 60%, about 55% to about 99%, about 55% to about 90%, about 55% to about 80%, about 55% to about 70%, about 55% to about 60%, about 60% to about 99%, about 60% to about 90%, about 60% to about 80%, about 60% to about 70%, about 65% to about 99%, about 65% to about 90%, about 65% to about 80%, about 65% to about 70%, about 70% to about 99%, about 70% to about 90%, about 70% to about 80%, about 75% to about 99%, about 75% to about 90%, about 75% to about 80%, about 80% to about 99%, about 80% to about 90%, about 85% to about 99%, about 85% to about 90%, about 90% to about 99%, about 90% to about 95%, or about 95% to about 99%. In another embodiment, the weight of silicone gum in the makeup composition is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99%.

In one embodiment in connection with the method, silicone resins are silicone materials which are formed by branched, cage-like oligosiloxanes with the general formula RₐSiX_{b}O_{c}, wherein R is a C₁-C₆alkyl group, C₂₋₆alkenyl group, or a phenyl group; X is H, OH, halogen, or C₁₋₁₀alkoxy; a, b, or c is a number independently ranging from 0 to 4. In another embodiment, silicone resins can be dissolved in a solvent, such as a polydimethylsiloxane or polyphenylsiloxane oil or a cyclomethicone. Silicone resins can be modified by an ester group, or by an alkyl group. In one embodiment, the silicone resin is a polymethylsilsequioxane.

In one embodiment in connection with the method, the silicone resins have a particle size ranging from about 0.001 μm to about 1000 μm, about 0.001 μm to about 900 μm, about 0.001 μm to about 800 μm, about 0.001 μm to about 700 μm, about 0.001 μm to about 600 μm, about 0.001 μm to about 500 μm, about 0.001 μm to about 400 μm, about 0.001 μm to about 300 μm, about 0.001 μm to about 200 μm, about 0.001 μm to about 100 μm, about 0.001 μm to about 90 μm, about 0.001 μm to about 80 μm, about 0.001 μm to about 70 μm, about 0.001 μm to about 60 μm, about 0.001 μm to about 50 μm, about 0.001 μm to about 40 μm, about 0.001 μm to about 30 μm, about 0.001 μm to about 20 μm, about 0.001 μm to about 10 μm, about 0.005 μm to about 1000 μm, about 0.005 μm to about 900 μm, about 0.005 μm to about 800 μm, about 0.005 μm to about 700 μm, about 0.005 μm to about 600 μm, about 0.005 μm to about 500 μm, about 0.005 μm to about 400 μm, about 0.005 μm to about 300 μm, about 0.005 μm to about 200 μm, about 0.005 μm to about 100 μm, about 0.005 μm to about 90 μm, about 0.005 μm to about 80 μm, about 0.005 μm to about 70 μm, about 0.005 μm to about 60 μm, about 0.005 μm to about 50 μm, about 0.005 μm to about 40 μm, about 0.005 μm to about 30 μm, about 0.005 μm to about 20 μm, about 0.005 μm to about 10 μm, about 0.01 μm to about 1000 μm, about 0.01 μm to about 900 μm, about 0.01 μm to about 800 μm, about 0.01 μm to about 700 μm, about 0.01 μm to about 600 μm, about 0.01 μm to about 500 μm, about 0.01 μm to about 400 μm, about 0.01 μm to about 300 μm, about 0.01 μm to about 200 μm, about 0.01 μm to about 100 μm, about 0.01 μm to about 90 μm, about 0.01 μm to about 80 μm, about 0.01 μm to about 70 μm, about 0.01 μm to about 60 μm, about 0.01 μm to about 50 μm, about 0.01 μm to about 40 μm, about 0.01 μm to about 30 μm, about 0.01 μm to about 20 μm, about 0.01 μm to about 10 μm, about 0.1 μm to about 1000 μm, about 0.1 μm to about 900 μm, about 0.1 μm to about 800 μm, about 0.1 μm to about 700 μm, about 0.1 μm to about 600 μm, about 0.1 μm to about 500 μm, about 0.1 μm to about 400 μm, about 0.1 μm to about 300 μm, about 0.1 μm to about 200 μm, about 0.1 μm to about 100 μm, about 0.1 μm to about 90 μm, about 0.1 μm to about 80 μm, about 0.1 μm to about 70 μm, about 0.1 μm to about 60 μm, about 0.1 μm to about 50 μm, about 0.1 μm to about 40 μm, about 0.1 μm to about 30 μm, about 0.1 μm to about 20 μm, about 0.1 μm to about 10 μm, about 0.5 μm to about 1000 μm, about 0.5 μm to about 900 μm, about 0.5 μm to about 800 μm, about 0.5 μm to about 700 μm, about 0.5 μm to about 600 μm, about 0.5 μm to about 500 μm, about 0.5 μm to about 400 μm, about 0.5 μm to about 300 μm, about 0.5 μm to about 200 μm, about 0.5 μm to about 100 μm, about 0.5 μm to about 90 μm, about 0.5 μm to about 80 μm, about 0.5 μm to about 70 μm, about 0.5 μm to about 60 μm, about 0.5 μm to about 50 μm, about 0.5 μm to about 40 μm, about 0.5 μm to about 30 μm, about 0.5 μm to about 20 μm, about 0.5 μm to about 10 μm, about 1 μm to about 1000 μm, about 1 μm to about 900 μm, about 1 μm to about 800 μm, about 1 μm to about 700 μm, about 1 μm to about 600 μm, about 1 μm to about 500 μm, about 1 μm to about 400 μm, about 1 μm to about 300 μm, about 1 μm to about 200 μm, about 1 μm to about 100 μm, about 1 μm to about 90 μm, about 1 μm to about 80 μm, about 1 μm to about 70 μm, about 1 μm to about 60 μm, about 1 μm to about 50 μm, about 1 μm to about 40 μm, about 1 μm to about 30 μm, about 1 μm to about 20 μm, about 1 μm to about 10 μm, about 2 μm to about 70 μm, about 3 μm to about 60 μm, about 4 μm to about 50 μm, about 5 μm to about 40 μm, about 5 μm to about 30 μm, about 5 μm to about 20 μm, about 5 μm to about 10 μm, or about 5 μm to about 8 μm.

In one embodiment in connection with the method, the weight of silicone resin in the makeup composition can range from about 1% to about 99%, about 1% to about 90%, about 1% to about 80%, about 1% to about 70%, about 1% to about 60%, about 1% to about 50%, about 1% to about 40%, about 1% to about 30%, about 1% to about 20%, about 1% to about 10%, about 1% to about 5%, about 5% to about 99%, about 5% to about 90%, about 5% to about 80%, about 5% to about 70%, about 5% to about 60%, about 5% to about 50%, about 5% to about 40%, about 5% to about 30%, about 5% to about 20%, about 5% to about 10%, about 10% to about 99%, about 10% to about 90%, about 10% to about 80%, about 10% to about 70%, about 10% to about 60%, about 10% to about 50%, about 10% to about 40%, about 10% to about 30%, about 10% to about 20%, about 15% to about 99%, about 15% to about 90%, about 15% to about 80%, about 15% to about 70%, about 15% to about 60%, about 15% to about 50%, about 15% to about 40%, about 15% to about 30%, about 15% to about 20%, about 20% to about 99%, about 20% to about 90%, about 20% to about 80%, about 20% to about 70%, about 20% to about 60%, about 20% to about 50%, about 20% to about 40%, about 20% to about 30%, about 25% to about 99%, about 25% to about 90%, about 25% to about 80%, about 25% to about 70%, about 25% to about 60%, about 25% to about 50%, about 25% to about 40%, about 25% to about 30%, about 30% to about 99%, about 30% to about 90%, about 30% to about 80%, about 30% to about 70%, about 30% to about 60%, about 30% to about 50%, about 30% to about 40%, about 35% to about 99%, about 35% to about 90%, about 35% to about 80%, about 35% to about 70%, about 35% to about 60%, about 35% to about 50%, about 35% to about 40%, about 40% to about 99%, about 40% to about 90%, about 40% to about 80%, about 40% to about 70%, about 40% to about 60%, about 40% to about 50%, about 45% to about 99%, about 45% to about 90%, about 45% to about 80%, about 45% to about 70%, about 45% to about 60%, about 45% to about 50%, about 50% to about 99%, about 50% to about 90%, about 50% to about 80%, about 50% to about 70%, about 50% to about 60%, about 55% to about 99%, about 55% to about 90%, about 55% to about 80%, about 55% to about 70%, about 55% to about 60%, about 60% to about 99%, about 60% to about 90%, about 60% to about 80%, about 60% to about 70%, about 65% to about 99%, about 65% to about 90%, about 65% to about 80%, about 65% to about 70%, about 70% to about 99%, about 70% to about 90%, about 70% to about 80%, about 75% to about 99%, about 75% to about 90%, about 75% to about 80%, about 80% to about 99%, about 80% to about 90%, about 85% to about 99%, about 85% to about 90%, about 90% to about 99%, about 90% to about 95%, or about 95% to about 99%. In another embodiment, the weight of silicone resin in the makeup composition is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99%.

In one embodiment in connection with the method, silicone compositions comprise silicone surfactants. The silicone surfactants comprise hydrophobic silicone and other functional groups as well. In one embodiment, a silicone surfactant is incorporated into a surface-active agent, with a polyoxyalkylene portion of the molecule and or a hydrocarbon portion of the molecule.

In one embodiment in connection with the method, the weight of silicone surfactants in the makeup composition can range from about 1% to about 99%, about 1% to about 90%, about 1% to about 80%, about 1% to about 70%, about 1% to about 60%, about 1% to about 50%, about 1% to about 40%, about 1% to about 30%, about 1% to about 20%, about 1% to about 10%, about 1% to about 5%, about 5% to about 99%, about 5% to about 90%, about 5% to about 80%, about 5% to about 70%, about 5% to about 60%, about 5% to about 50%, about 5% to about 40%, about 5% to about 30%, about 5% to about 20%, about 5% to about 10%, about 10% to about 99%, about 10% to about 90%, about 10% to about 80%, about 10% to about 70%, about 10% to about 60%, about 10% to about 50%, about 10% to about 40%, about 10% to about 30%, about 10% to about 20%, about 15% to about 99%, about 15% to about 90%, about 15% to about 80%, about 15% to about 70%, about 15% to about 60%, about 15% to about 50%, about 15% to about 40%, about 15% to about 30%, about 15% to about 20%, about 20% to about 99%, about 20% to about 90%, about 20% to about 80%, about 20% to about 70%, about 20% to about 60%, about 20% to about 50%, about 20% to about 40%, about 20% to about 30%, about 25% to about 99%, about 25% to about 90%, about 25% to about 80%, about 25% to about 70%, about 25% to about 60%, about 25% to about 50%, about 25% to about 40%, about 25% to about 30%, about 30% to about 99%, about 30% to about 90%, about 30% to about 80%, about 30% to about 70%, about 30% to about 60%, about 30% to about 50%, about 30% to about 40%, about 35% to about 99%, about 35% to about 90%, about 35% to about 80%, about 35% to about 70%, about 35% to about 60%, about 35% to about 50%, about 35% to about 40%, about 40% to about 99%, about 40% to about 90%, about 40% to about 80%, about 40% to about 70%, about 40% to about 60%, about 40% to about 50%, about 45% to about 99%, about 45% to about 90%, about 45% to about 80%, about 45% to about 70%, about 45% to about 60%, about 45% to about 50%, about 50% to about 99%, about 50% to about 90%, about 50% to about 80%, about 50% to about 70%, about 50% to about 60%, about 55% to about 99%, about 55% to about 90%, about 55% to about 80%, about 55% to about 70%, about 55% to about 60%, about 60% to about 99%, about 60% to about 90%, about 60% to about 80%, about 60% to about 70%, about 65% to about 99%, about 65% to about 90%, about 65% to about 80%, about 65% to about 70%, about 70% to about 99%, about 70% to about 90%, about 70% to about 80%, about 75% to about 99%, about 75% to about 90%, about 75% to about 80%, about 80% to about 99%, about 80% to about 90%, about 85% to about 99%, about 85% to about 90%, about 90% to about 99%, about 90% to about 95%, or about 95% to about 99%. In another embodiment, the weight of silicone surfactants in the makeup composition is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99%.

In one embodiment in connection with the method, the silicone is selected from dimethicone, methicone, trimethicone, cyclomethicone, amodimethicone, trimethylsilylamodimethicone, cyclotetrasiloxane, cyclopentasiloxane, cyclehexasiloxane, polydimethylsiloxane, polymethylsilsequioxane, dimethiconol, and mixtures thereof.

In one embodiment in connection with the method, the makeup composition further comprises a coloring agent. In one embodiment, the coloring agent comprises pigments, nacres, and reflective particles, dyestuffs, and mixtures thereof.

In another embodiment in connection with the method, the pigments comprise white or colored, mineral or organic particles of any shape, which are insoluble in a physiological medium, and which are intended to color the composition. In some embodiments, the pigments are white.

In other embodiments in connection with the method, the mineral pigments comprise titanium dioxide, such as pigmentary titanium dioxide rutile type, optionally surface-treated, zirconium oxide, cerium oxide, zinc oxide, iron (black, yellow or red) oxide or chromium oxide, manganese violet, ultramarine blue, chromium hydrate, ferric blue, and metal powders, for instance aluminum powder and copper powder.

In another embodiment in connection with the method, the organic pigments may be chosen from carbon black, pigments of D & C type, and lakes based on cochineal carmine, barium, strontium, calcium, and aluminum, organic pigments of azo dyes, anthraquinone dyes, indigoid dyes, xanthene dyes, pyrene dyes, quinoline dyes, triphenylmethane dyes and fluorane dyes.

In one embodiment in connection with the method, the dyes are water-soluble dyes. Such dyes may include FD&C Blue No. 11, FD&C Blue No. 12, FD&C Green No. 13, FD&C Red No. 13, FD&C Red No. 140, FD&C Yellow No. 15, FD&C Yellow No. 16, D&C Blue No. 14, D&C Blue No. 19; D&C Green No. 15, D&C Green No. 16, D&C Green No. 18, D&C Orange No. 14, D&C Orange No. 15, D&C Orange No. 110, D&C Orange No. 111, D&C Orange No. 1 17, FD&C Red No. 14, D&C Red No. 16, D&C Red No. 17, D&C Red No. 18, D&C Red No. 19, D&C Red No. 117, D&C Red No. 119, D&C Red No. 121, D&C Red No. 122, D&C Red No. 127, D&C Red No. 128, D&C Red No. 130, D&C Red No. 131, D&C Red No. 134, D&C Red No. 139, FD&C Red No. 140, D&C Violet No. 12, D&C Yellow No. 17, Ext. D&C Yellow No. 17, D&C Yellow No. 18, D&C Yellow No. 11-1, D&C Brown No. 11, Ext. D&C Violet No. 12, D&C Blue No. 16 and D&C Yellow No. 110. The above dyes are well known, commercially available materials, with their chemical structure being described, e.g., in 21 C. F. R. Part 74 (as revised Apr. 1, 1988) and in the CTFA Cosmetic Ingredient Handbook, (1988), published by the Cosmetics, Toiletry and Fragrances Association, Inc. These publications are incorporated herein by reference.

The chemical materials corresponding to each of the organic dyestuffs mentioned above are recited in the publication “International Cosmetic Ingredient Dictionary and Handbook”, 1997 edition, pages 371 to 386 and 524 to 528, published by The Cosmetic, Toiletries and Fragrance Association, the content of which is incorporated into the present patent application by reference.

In another embodiment in connection with the method, the nacres comprise colored particles of any form, which may or may not be iridescent, especially produced by certain mollusks in their shell, or alternatively synthesized, and which have a color effect via optical interference.

Examples of nacres include, but are not limited to, nacreous pigments such as titanium mica coated with an iron oxide, mica coated with bismuth oxychloride, titanium mica coated with chromium oxide, and nacreous pigments based on bismuth oxychloride. They may also be mica particles at the surface of which are superimposed at least two successive layers of metal oxides and/or of organic dyestuffs. The nacres may more particularly have a yellow, pink, red, bronze, orange, brown, gold and/or coppery color or glint.

As illustrations of nacres that may be introduced into the composition, the nacres include, but are not limited to, the gold-colored nacres sold especially by the company Engelhard under the name Brilliant gold 212G (Timica), Gold 222C (Cloisonne), Sparkle gold (Timica), Gold 4504 (Chromalite) and Monarch gold 233X (Cloisonne); the bronze nacres sold especially by the company Merck under the name Bronze fine (17384) (Colorona) and Bronze (17353) (Colorona) and by the company Engelhard under the name Super bronze (Cloisonne); the orange nacres sold especially by the company Engelhard under the name Orange 363C (Cloisonne) and Orange MCR 101 (Cosmica) and by the company Merck under the name Passion orange (Colorona) and Matte orange (17449) (Microna); the brown nacres sold especially by the company Engelhard under the name Nu-antique copper 340XB (Cloisonne) and Brown CL4509 (Chromalite); the nacres with a copper tint sold especially by the company Engelhard under the name Copper 340A (Timica); the nacres with a red tint sold especially by the company Merck under the name Sienna fine (17386) (Colorona); the nacres with a yellow tint sold especially by the company Engelhard under the name Yellow (4502) (Chromalite); the red nacres with a gold tint sold especially by the company Engelhard under the name Sunstone G012 (Gemtone); the pink nacres sold especially by the company Engelhard under the name Tan opale G005 (Gemtone); the black nacres with a gold tint sold especially by the company Engelhard under the name Nu antique bronze 240 AB (Timica), the blue nacres sold especially by the company Merck under the name Matte blue (17433) (Microna), the white nacres with a silvery tint sold especially by the company Merck under the name Xirona Silver, and the golden-green pink-orange nacres sold especially by the company Merck under the name Indian summer (Xirona), and mixtures thereof.

Further, the nacres may comprise a borosilicate substrate coated with titanium oxide or polyethylene terephthalate flakes.

In another embodiment in connection with the method, the reflective particles comprise particles whose size, structure, especially the thickness of the layer(s) of which they are made and their physical and chemical nature, and surface state, allow them to reflect incident light. This reflection may, where appropriate, have an intensity sufficient to create at the surface of the composition or of the mixture, when it is applied to the support to be made up, points of overbrightness that are visible to the naked eye, i.e. more luminous points that contrast with their environment by appearing to sparkle.

In another embodiment in connection with the method, the reflective particles may be selected so as not to significantly alter the coloration effect generated by the coloring agents with which they are combined, and more particularly so as to optimize this effect in terms of color yield. They may more particularly have a yellow, pink, red, bronze, orange, brown, gold and/or coppery color or tint. These particles may have varied forms and may especially be in platelet or globular form, in particular in spherical form.

In another embodiment in connection with the method, the reflective particles, whatever their form, may or may not have a multilayer structure and, in the case of a multilayer structure, may have, for example, at least one layer of uniform thickness, in particular of a reflective material.

In one embodiment, when the reflective particles do not have a multilayer structure, they may be composed, for example, of metal oxides, especially titanium or iron oxides obtained synthetically.

In another embodiment, when the reflective particles have a multilayer structure, they may comprise, for example, a natural or synthetic substrate, especially a synthetic substrate at least partially coated with at least one layer of a reflective material, especially of at least one metal or metallic material. The substrate may be made of one or more organic and/or inorganic materials.

More particularly, they may be selected from glasses, ceramics, graphite, metal oxides, aluminas, silicas, silicates, especially aluminosilicates and borosilicates, and synthetic mica, and mixtures thereof.

Examples of reflective particles comprises a mineral substrate coated with a layer of metal, mention may also be made of particles comprising a silver-coated borosilicate substrate.

In one embodiment, the reflective materials may comprise a layer of metal or of a metallic material.

In another embodiment in connection with the method, the weight of the coloring agent in the makeup composition can range from about 0.01% to about 30%, about 0.01% to about 25%, about 0.01% to about 20%, about 0.01% to about 15%, about 0.01% to about 10%, about 0.01% to about 5%, about 0.01% to about 3%, about 0.01% to about 2%, about 0.01% to about 1%, about 0.01% to about 0.5%, about 0.1% to about 30%, about 0.1% to about 25%, about 0.1% to about 20%, about 0.1% to about 15%, about 0.1% to about 10%, about 0.1% to about 5%, about 0.1% to about 3%, about 0.1% to about 2%, about 0.1% to about 1%, about 0.1% to about 0.5%, about 0.5% to about 30%, about 0.5% to about 25%, about 0.5% to about 20%, about 0.5% to about 15%, about 0.5% to about 10%, about 0.5% to about 5%, about 0.5% to about 3%, about 0.5% to about 2%, about 0.5% to about 1%, about 1% to about 30%, about 1% to about 25%, about 1% to about 20%, about 1% to about 15%, about 1% to about 10%, about 1% to about 5%, about 1% to about 3%, about 1% to about 2%, about 2% to about 30%, about 5% to about 30%, about 5% to about 25%, about 5% to about 20%, about 5% to about 15%, about 5% to about 10%, about 10% to about 30%, about 10% to about 25%, about 10% to about 20%, about 15% to about 30%, about 15% to about 25%, about 15% to about 20%, about 20% to about 30%, about 20% to about 25%, about 25% to about 30%, or about 25% to about 28%.

In another embodiment in connection with the method, the makeup composition simultaneously conceals and treats said skin defect when applied to the skin. In one embodiment, the extent of concealment is measured in terms of the “hiding power” or “covering power” of the material. The makeup composition coverage can be tested by various methods known in the art and/or described herein. A standard test for measuring the hiding power of materials that dry to form a film, for example, is the Dry-Film Coverage Test Method based on ASTM D 6441-05. See ASTM International Designation: D 6441-05 “Standard Test Methods for Measuring the Hiding Power of Powder Coatings,” and herein incorporated in its entirety by reference.

In one embodiment in connection with the method, the following formula can be used to measure the extent of concealment: Contrast ratio (Coverage) (Cw)=100*Ro/Rw; where Rw refers to reflectance of the applied test makeup composition over a white substrate; Ro refers to reflectance of the applied test makeup composition over a black substrate, and where the luminous reflectance factor is measured by a Konica Minolta handheld spectrophotometer. The method involves drawing down a sample on a polyester film and a control mil thickness; waiting for 15 minutes for samples to dry; putting the polyester upon a LENETA card and measuring reflectance (L* value), Ro (black background) and Rw (white background) using the spectrophotometer. Each LENETA card can read 3 data points on the black background and this can be repeated for 6 different LENETA cards. The 18 data points can be collected and the contrast ratio determined. For example, contrast ratio can be based on an average of these data points. The coverage values then can be normalized based on % solids in the formula to account for differences in film thickness. A high contrast ratio translates into a film with high coverage. Some embodiments of the disclosure herein provide makeup composition coverage ranging from about 30 to about 200, about 40 to about 200, about 50 to about 200, about 60 to about 200, about 70 to about 200, about 80 to about 200, about 90 to about 200, about 100 to about 200, about 100 to about 190, about 100 to about 180, about 100 to about 170, about 100 to about 160, about 100 to about 150, about 100 to about 140, about 100 to about 130, about 100 to about 120, or about 100 to about 110. Preferably, the coverage of the makeup compositions of the disclosure herein is about 30, about 50, about 80, about 100, about 150, or about 200.

In another embodiment in connection with the method, the makeup composition of the disclosure herein counteracts the phylogenetic process by which scars are formed. In general, epidermis may take weeks to sufficiently regenerate after a full-thickness wound has occurred due to penetration through the epidermis and into the dermis layers. The new immature stratum corneum allows abnormally high levels of transepidermal water loss. This dehydration of the stratum corneum signals the keratinocytes to produce cytokines, which signal to fibroblasts to synthesize and release collagen. Naturally, wound healing has been optimized for speed as opposed to quality of healing under far less sanitary conditions to prevent infection. This speed optimization produces excess collagen which rushes to the scar site and is the cause of many undesirable physical and aesthetic properties associated with scarring.

The makeup compositions of the disclosure herein provide improved occlusion and hydration to the wound bed. They aid the newly formed, underdeveloped stratum corneum in retaining optimum water levels. Unlike other dressings, the makeup compositions of the disclosure herein provide a level of occlusion similar to normal skin and significantly reduce collagen formation in the scar site. Therefore, stratum corneum hydration is normalized and undesirable physical and aesthetic properties associated with scarring are minimized. In a preferred embodiment, the makeup composition comprises a substantial amount of silicone.

In another embodiment in connection with the method, the makeup compositions of the disclosure herein generate a negatively charged static electric field through the creation of friction between it and the skin. This static electricity is thought to aid the alignment of collagen cells, thus resulting in the involution of raised scars.

In another embodiment in connection with the method, the makeup compositions of the disclosure herein inhibit the body's natural reaction to increase skin capillaries through hyperemia. This reduces the blood supply to the scar site and the exaggeration of the healing process, along with the intensity of the fully formed scar's appearance and physical properties.

The method of the disclosure herein should not be limited to any mechanism described herein.

In another embodiment in connection with the method, the skin defect is a scar. In one embodiment, the scar is selected from a keloid scar, a hypertrophic scar, a contracture scar, an acne scar, and a surgical scar. In another embodiment, the scar is a surgical scar following suture removal about 7 to about 14 days after surgery. Preferably, the scar is a surgical scar following suture removal about 7 days after surgery, about 8 days after surgery, about 9 days after surgery, about 10 days after surgery, about 11 days after surgery, about 12 days after surgery, about 13 days after surgery, or about 14 days after surgery.

In another embodiment in connection with the method, the makeup compositions further provide UV protection. In one embodiment, the makeup composition screens out both UV-B and UV-A radiation. In one embodiment, the makeup composition has a sun protection factor (SPF) of from about 4 to about 60, about 5 to about 60, about 6 to about 60, about 7 to about 60, about 8 to about 60, about 9 to about 60, about 10 to about 60, about 10 to about 50, about 10 to about 40, about 10 to about 30, about 10 to about 25, about 10 to about 20, about 10 to about 19, about 10 to about 18, about 10 to about 17, about 10 to about 16, or about 10 to about 15. In another embodiment, the makeup composition has a sun protection factor (SPF) of about 5, about 10, about 15, about 20, about 25, about 30, about 35, about 40, about 45, about 50, about 55, or about 60.

In another embodiment in connection with the method, the makeup composition further comprises one or more sunscreen agents. The suitable sunscreen agents are selected from aminobenzoic acid, amyldimethyl PABA, cinoxate, diethanolaminep-methoxycinnamate, digalloyl trioleate, dioxybenzone, 2-ethoxyethyl p-methoxycinnamate, ethyl 4-bis(hydroxypropyl)aminobenzoate, 2-ethylhexyl-2-cyano-3,3-diphenylacrylate, ethylhexyl p-methoxycinnamate, 2-ethylhexyl salicylate, glyceryl aminobenzoate, homomenthyl salicylate, homosalate, 3-imidazol-4-ylacrylic acid and ethyl ester, methyl anthranilate, octyldimethyl PABA, 2-phenylbenzimidazole-5-sulfonic acid and salts, red petrolatum, sulisobenzone, titanium dioxide, triethanolamine salicylate, N,N,N-trimethyl-4-(2-oxoborn-3-ylidene methyl)anillinium methyl sulfate, and mixtures thereof.

Suitable sunscreens agents active in the UV-A and/or UV-B range further comprise p-aminobenzoic acid, oxyethylene (25 mol) p-aminobenzoate, 2-ethylhexyl p-dimethylaminobenzoate, ethyl N-oxypropylene p-aminobenzoate, glycerol p-aminobenzoate, 4-isopropylbenzyl salicylate, 2-ethylhexyl 4-methoxycinnamate, methyl diisopropylcinnamate, isoamyl 4-methoxycinnamate, diethanolamine 4-methoxycinnamate, 3-(4′-trimethylammunium)-benzyliden-bornan-2-one methylsulfate, 2-hydroxy-4-methoxybenzophenone, 2-hydroxy-4-methoxybenzophenone-5-sulfonate, 2,4-dihydroxybenzophenone, 2,2′,4,4′-tetrahydroxybenzophenone, 2,2′-dihydroxy-4,4′-dimethoxybenzophenone, 2-hydroxy-4-n-octoxybenzophenone, 2-hydroxy-4-methoxy-4′-methoxybenzophenone-(2-oxobom-3-ylidene)-tolyl-4-sulfonic acid and soluble salts thereof, 3-(4′-sulfo)benzyliden-bornan-2-one and soluble salts thereof, 3-(4′-methylbenzylidene)-d,l-camphor, 3-benzylidene-d,l-camphor, benzene 1,4-di(3-methylidene-10-camphosulfonic) acid and salts thereof (the product Mexoryl SX described in U.S. Pat. No. 4,585,597 issued to Lange et al. on Apr. 29, 1986), urocanic acid, 2,4,6-tris[p-(2′-ethylhexyl-1′-oxycarbonyl)-anilino]-1,3,5-triazine, 2-[(p-(tertiobutylamido)anilino]-4,6-bis-[(p-(2′-ethylhexyl-1′-oxycarbonyl)anilino]-1,3,5-triazine, 2,4-bis {[4-(2-ethyl-hexyloxy)]-2-hydroxy]-phenyl}-6-(4-methoxy-phenyl)-1,3,5-triazine (“TINOSORB S” marketed by Ciba), the polymer of N-(2 et 4)-[(2-oxoborn-3-yliden)methyl]benzyl]-acrylamide, 1,4-bisbenzimidazolyl-phenylen-3,3′,5,5′-tetrasulfonic acid and salts thereof, the benzalmalonate-substituted polyorganosiloxanes, the benzotriazole-substituted polyorganosiloxanes (Drometrizole Trisiloxane), dispersed 2,2′-methylene-bis-[6-(2H-benzotriazol-2-yl)-4-(1,1,3,3-tetramethylbutyl)phenol] such as that marketed under the trademark MIXXIM BB/100 by Fairmount Chemical, or micronized in dispersed form thereof such as that marketed under the trademark TINOSORB M by Ciba-Geigy, and solubilized 2,2′-methylene-bis-[6-(2H-benzotriazol-2-yl)-4-(methyl)phenol] such as that marketed under the trademark MIXXIM BB/200 by Fairmount Chemical. Typically combinations of one of more of these sunscreens are used, the dibenzoyl methane derivatives other than avobenzone are described, for example, in FR-2,326,405, FR-2,440,933, and EP-0,114,607, hereby expressly incorporated by reference.

Other dibenzoyl methane sunscreens other than avobenzone comprise 2-methyl dibenzoylmethane, 4-methyldibenzoylmethane, 4-isopropyldibenzoylmethane, 4-tert.-butyldibenzoylmethane, 2,4-dimethyldibenzoylmethane, 2,5-dimethyldibenzoylmethane, 4,4′-diisopropyldibenzoylmethane, 4,4′-dimethoxydibenzoylmethane, 2-methyl-5-isopropyl-4′-methoxydibenzoylmethane, 2-methyl-5-tert.-butyl-4′-methoxydibenzoylmethane, 2,4-dimethyl-4′-methoxydibenzoylmethane, 2,6-dimethyl-4-tert.-butyl-4′-methoxydibenzoylmethane.

In another embodiment in connection with the method, the makeup compositions may further comprise a moisturizer, emollient, humectant, thickener, skin toner, or a mixture thereof.

In another embodiment in connection with the method, the compositions according to the disclosure are homogeneous in general. A homogeneous distribution of the constituents is observed. In one embodiment, the compositions are wax-in-oil mixtures, which are a homogeneous and clear mixture under warm conditions and a homogeneous dispersion of the wax in the oil is formed during cooling. In another embodiment, good dispersion of the pigments is observed. In one embodiment, good cohesion of the product is observed.

In another embodiment, the makeup compositions of the disclosure herein remain homogeneous even after application to the skin and/or mucous membranes, this being the case for at least several hours.

In another embodiment, the makeup compositions of the disclosure herein also exhibit excellent cosmetic properties. They are soft, they adhere sufficiently but not too much to the skin and/or mucous membranes. They are readily applied and they provide a very pleasant feeling on application. In particular, as regards the cosmetic powders, they are easy to compact, disintegrate well and exhibit good hardness.

In another embodiment in connection with the method, the scar is substantially healed after the treatment. In another embodiment in connection with the method, the treatment results in substantial reduction in the amount and visibility of scar tissue.

In another embodiment in connection with the method, the makeup compositions further provide a calming or soothing effect to the skin. In another embodiment in connection with the method, the makeup compositions further comprise a calming agent. Calming agents may include, but are not limited to, vitamin A palmitate; Phytelene Complex EGX 244, which is a botanical blend of extracts of calendula, chamomile, linden, cornflower, matricaria and hypericum; allantoin; dipotassium glycyrrhizinate; stearyl glycyrrhizinate; bisabolo((3-cyclohexene-1-methanol-∝,4-dimethyl-∝(4-methyl-3-pentenyl)); squalane NF; cetyl ester wax; shea butter; orange roughy oil; hydrogenated phospholipids, and HETESTER™ FAO, which is a C₁₂C₁₅ alcohol octanoate, available from Bernel Chemical Company, Englewood, N.J.

In another embodiment in connection with the method, the scar is substantially healed after the treatment. In another embodiment, the treatment results in substantial reduction in the amount and visibility of scar tissue. In one embodiment, the size of the scar is reduced at least 10%, at least 20%, at least 30%, at least 40%, at least 50% after the treatment. The size of the scar can be measured based on the “scar elevation index” under 100× magnification. The scar elevation index is calculated as the area of the scar dermis (S) divided by the normal area of dermis (N), the latter being based on the height of the dermis in adjacent, unwounded skin (R. Reid et al., J Plast Reconstr Aesthet Surg. (2007), 60:64-72).

In another embodiment in connection with the method, the volume of the makeup composition applied to the skin defect is about 0.1 to about 10 mL, about 0.2 to about 10 mL, about 0.3 to about 10 mL, about 0.4 to about 10 mL, about 0.5 to about 10 mL, about 0.6 to about 10 mL, about 0.7 to about 10 mL, about 0.8 to about 10 mL, about 0.9 to about 10 mL, about 1 to about 10 mL, about 2 to about 10 mL, about 3 to about 10 mL, about 4 to about 10 mL, about 5 to about 10 mL, about 6 to about 10 mL, about 7 to about 10 mL, about 8 to about 10 mL, or about 9 to about 10 mL. In another embodiment in connection with the method, the volume of the makeup composition applied to the skin defect is about 0.1 mL, about 0.2 mL, about 0.3 mL, about 0.4 mL, about 0.5 mL, about 0.6 mL, about 0.7 mL, about 0.8 mL, about 0.9 mL, about 1 mL, about 2 mL, about 3 mL, about 4 mL, about 5 mL, about 6 mL, about 7 mL, about 8 mL, about 9 mL, or about 10 mL.

In another embodiment in connection with the method, the viscosity of the makeup composition is about 1,000 cP to about 1,000,000 cP. In one embodiment, the dynamic viscosity of the makeup compositions in the disclosure herein at 25° C. may be measured by means of a rotational viscometer of Mettler RM 180 type. The Mettler RM 180 instrument (Rheomat) may be equipped with different spindles as a function of the size order of the viscosity to be measured. For a viscosity varying from 180 to 4,020 cP, the instrument is equipped with a spindle 3. For a viscosity varying from 1,000 to 24,000 cP, the instrument is equipped with a spindle 4, and for a viscosity varying from 8,000 to 122,000 cP the instrument is equipped with a spindle 5.

The viscosity is read off in the instrument in deviation units (DU). Tables supplied with the measuring instrument then allow the corresponding value in poises to be obtained. The rotational speed of the spindle is approximately 200 revolutions/minute.

When the spindle is set in rotation, at a constant driven rotational speed (in this case 200 revolutions/minute), the viscosity value of the composition may vary over time until it reaches a constant value. Measurements are taken at regular intervals until constant measurement values are obtained. The viscosity value which has become constant over time is the value taken as being the dynamic viscosity value of the composition according to the disclosure herein. According to the 75 measurement system, the viscosity measurement is taken after 10 minutes.

In another embodiment in connection with the method, the viscosity of the makeup composition varies in particular from about 1,000 to about 1,000,000 cP, about 1,000 to about 900,000 cP, about 1,000 to about 800,000 cP, about 1,000 to about 700,000 cP, about 1,000 to about 600,000 cP, about 1,000 to about 500,000 cP, about 1,000 to about 500,000 cP, about 1,000 to about 400,000 cP, about 1,000 to about 300,000 cP, about 1,000 to about 250,000 cP, about 1,000 to about 200,000 cP, about 1,000 to about 150,000 cP, about 1,000 to about 125,000 cP, about 1,000 to about 100,000 cP, about 1,000 to about 62,500 cP, about 1,000 to about 50,000 cP, about 1,000 to about 32,000 cP, about 1,000 to about 16,000 cP, about 1,000 to about 10,000 cP, about 1,000 to about 8,000 cP, about 1,000 to about 7,000 cP, about 1,000 to about 6,000 cP, about 1,000 to about 5,000 cP, about 1,000 to about 4,000 cP, about 1,000 to about 3,000 cP, or about 1,000 to about 2,000 cP.

In another embodiment in connection with the method, the makeup composition further comprises a gelling agent. The gelling agent modifies the viscosity of the makeup composition.

In another embodiment in connection with the method, the makeup compositions comprise at least one liquid-fatty-phase gelling agent. In one embodiment, the liquid-fatty-phase gelling agent is an inorganic gelling agent. In one specific embodiment, the liquid-fatty-phase gelling agent is a non-crystalline, non-silicone gelling agent. Particularly, the liquid-fatty-phase gelling agents which can be used in the makeup compositions herein are selected from agents which gel via chemical crosslinking and agents which gel via physical crosslinking.

In another embodiment in connection with the method, the liquid-fatty-phase gelling agents by chemical crosslinking are selected from polyorganosiloxanes comprising R₂SiO and RSiO_{1.5} units and optionally R₃SiO_{0.5} and/or SiO₂ units, in which the radicals R are selected, independently of one another, from a hydrogen, an alkyl such as methyl, ethyl or propyl, an aryl such as phenyl or tolyl, an unsaturated aliphatic group such as vinyl, the weight ratio of the R₂SiO units to the RSiO₁₅ units ranging from 1/1 to 30/1.

Polyorganosiloxanes which are insoluble and swellable in silicone oil, obtained by addition of a polyorganohydrosiloxane (1) and a polyorganosiloxane (2) having unsaturated aliphatic groups such that the amount of hydrogen or of unsaturated aliphatic groups in (1) and (2) respectively ranges from 1 to 20 mol % when the polyorganosiloxane is non-cyclic and from 1 to 50 mol % when the polyorganosiloxane is cyclic. These polyorganosiloxanes may optionally contain from 1 to 40 oxyalkylene groups, such as oxypropylene and/or oxyethylene groups.

Examples of polyorganosiloxanes of the disclosure herein include, but are not limited to, KSG6 by Shin-Etsu, Trefil E-505C or Trefil E-506C from Dow Corning, Gransil from Grant Industries (SR-CYC, SR DMF10, SR-DC556) or those sold in the form of preconstituted gels KSG15, KSG17, KSG16, KSG18 and KSG21 from Shin-etsu, Gransil SR 5CYC gel, Gransil SR DMF 10 gel, Gransil SR DC556 gel, SF 1204 and JK 113 from General Electric, or a mixture of thereof.

In another embodiment in connection with the method, the liquid-fatty-phase gelling agents by physical crosslinking are selected from gelling agents by molecular agitation, hydrogen interactions, dipolar interactions, or a mixture of thereof.

In another embodiment in connection with the method, the makeup composition comprises silicone and color agents. In one embodiment, the ratio between silicone and color agents ranges from about 0.01 to about 99, about 0.1 to about 95, about 1 to about 90, about 5 to about 85, about 10 to about 80, about 15 to about 75, about 20 to about 70, about 25 to about 65, about 30 to about 60, about 35 to about 55, about 40 to about 50, or about 40 to about 45. In another embodiment, the ratio between silicones and color agents is about 0.01, about 0.05, about 0.1, about 0.5, about 1, about 5, about 10, about 15, about 20, about 25, about 30, about 35, about 40, about 45, about 50, about 55, about 60, about 65, about 70, about 75, about 80, about 85, about 90, about 95, or about 99.

In another embodiment in connection with the method, the makeup composition of the disclosure herein is maintained on the skin for at least 1-12 hours, at least 1-11 hours, at least 1-10 hours, at least 1-9 hours, at least 1-8 hours, at least 1-7 hours, at least 1-6 hours, at least 1-5 hours, at least 1-4 hours, or at least 1-3 hours. In another embodiment in connection with the method, the makeup composition of the disclosure herein is maintained on the skin for about 1 hours, about 2 hours, about 3 hours, about 4 hours, about 5 hours, about 6 hours, about 7 hours, about 8 hours, about 9 hours, about 10 hours, about 11 hours, or about 12 hours.

In another embodiment in connection with the method, the makeup composition of the disclosure herein is applied daily for about 5 days to about 120 days, about 5 days to about 110 days, about 5 days to about 100 days, about 5 days to about 90 days, about 5 days to about 80 days, about 5 days to about 70 days, about 5 days to about 60 days, about 5 days to about 50 days, about 5 days to about 40 days, about 5 days to about 30 days, about 5 days to about 20 days, about 5 days to about 10 days, about 5 days to about 7 days, about 10 days to about 120 days, about 10 days to about 110 days, about 10 days to about 100 days, about 10 days to about 90 days, about 10 days to about 80 days, about 10 days to about 70 days, about 10 days to about 60 days, about 10 days to about 50 days, about 10 days to about 40 days, about 10 days to about 30 days, about 10 days to about 20 days, about 20 days to about 120 days, about 20 days to about 110 days, about 20 days to about 100 days, about 20 days to about 90 days, about 20 days to about 80 days, about 20 days to about 70 days, about 20 days to about 60 days, about 20 days to about 50 days, about 20 days to about 40 days, about 20 days to about 30 days, about 30 days to about 120 days, about 40 days to about 120 days, about 50 days to about 120 days, about 60 days to about 120 days, about 70 days to about 120 days, about 80 days to about 120 days, about 90 days to about 120 days, about 100 days to about 120 days, or about 110 days to about 120 days.

In another embodiment in connection with the method, the makeup composition of the disclosure herein is topically applied to the skin as needed. Typically, the composition is topically applied to the skin one to five times per day. In one embodiment, the composition is topically applied to the skin one, two, three, four, or five times per day. However, application of a present composition can be more or less frequent as prescribed, required, or desired.

In another embodiment in connection with the method, the subject of the disclosure herein is a mammal. In a preferred embodiment, the subject is a human. In another embodiment, the human is a human male. In another embodiment, the human is an human female. In another embodiment, the human is at the age of above 18.

In some embodiments in connection with the method, the method is for veterinary use. In a preferred embodiment, the subject is a mammal such as a non-primate, e.g., rabbit, cow, pig, horse, cat, dog, rat, and a primate, e.g., a monkey, such as a Cynomolgous monkey.

In another embodiment in connection with the method, the makeup compositions of the disclosure herein are applied to the skin by spraying, spreading, rubbing, or brushing. The preferred route of administration is by rubbing the makeup composition onto the skin with a soft massage to ensure intimate contact with the skin.

In another embodiment in connection with the method, the makeup compositions contain less than 1% or none of comedogenic compounds. Comedogenic compounds used herein refer to ingredients tending to clog pores of the skin. In one embodiment, the makeup compositions contain less than 1%, or does not contain any of the following compounds: sodium chloride, wheat germ oil, sodium lauryl sulfate, red algae, octyl stearate, laureth 4, isopropyl myristate, isopropyl isostearate, isocetyl stearate, carrageenan, algae extract, or combinations thereof.

### Makeup Compositions

In another aspect, the present disclosure provides a makeup composition comprising at least 50%, 55%, 60%, 65%, 70%, 75%, 80%, 85%, 90%, or 95% by weight of silicone and a coloring agent, wherein the composition, when applied to a scar on the skin of a subject, treats said scar on the skin of the subject while simultaneously camouflaging the scar and providing UV protection.

In one embodiment in connection with the makeup composition, the silicone refers broadly to organosilicon polymers or oligomers with a linear or cyclic, branched or crosslinked structure, of variable molecular weight, obtained by polymerization and/or polycondensation of suitably functionalized silanes and essentially composed of a repetition of main units in which the silicon atoms are connected to one another via oxygen atoms (siloxane bond ≡Si—O—Si≡), optionally substituted hydrocarbonaceous radicals being directly bonded via carbon atom to the said silicon atoms. The commonest hydrocarbonaceous radicals are alkyl radicals, in particular (C₁-C₃₀) alkyl radicals and especially methyl radicals, fluoroalkyl radicals or aryl radicals, in particular the phenyl radical.

A variety of silicone compounds can be used in the composition. The silicone compound can be chosen from silicone liquid, silicone oils, which may or may not be volatile, silicone waxes, silicone gums, silicone resins, silicone surfactants and/or their mixtures.

In another embodiment in connection with the makeup composition, silicone oil is a compound corresponding to the following general formula (I):

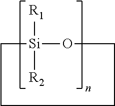

wherein R₁ and R₂ are an independent C₁₋₆ alkyl;
n is in integer from 3-100.

In one embodiment, silicone oil is linear polydimethylsiloxanes (PDMSs) corresponding to the following general formula (II):

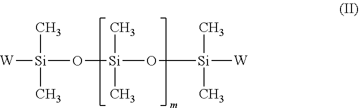

wherein

W is —CH₃ or —OH, and

m is an integer from 0 to 2000.

In one embodiment in connection with the makeup composition, the weight of silicone oil in the makeup composition is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%.

In one embodiment in connection with the makeup composition, the silicone wax is a silicone product which is a semi-solid or solid at room temperature. In another embodiment, the silicone wax has a melting point ranging from about 25 to about 135° C.

In one embodiment in connection with the method, the silicone wax is a compound corresponding to the following general formula (III):

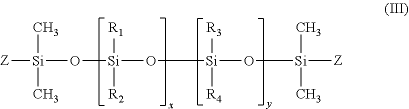

wherein
R₁, R₂, R₃, and R₄ are independently selected from C₁-C₃₀ alkyl, C₁-C₃₀ alkoxy, and aryl, wherein each group is optionally substituted by an ester group;
Z is selected from C₁-C₃₀ alkyl, C₁-C₃₀ alkoxy, C₁-C₃₀ ester, and aryl; and
x and y are independently an integer from 0 to 100.

In one embodiment in connection with the makeup composition, the weight of silicone wax in the makeup composition is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%.

In one embodiment in connection with the makeup composition, the silicone gum is a compound with a viscosity at 25° C., ranging from 500,000 cSt to 5×10⁷ cSt. In another embodiment, the silicone gum does not exhibit a melting point.

In another embodiment in connection with the makeup composition, the silicone gum is a compound corresponding to the following general formula (IV):

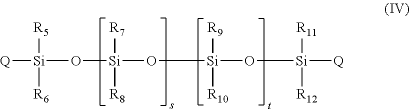

wherein
R₅, R₆, R₇, R₈, R₉, R₁₀, R₁₁, and R₁₂ are independently an C₁-C₆ alkyl;
Q is selected from C₁-C₆ alkyl, —OH, and C₂₋₁₀alkenyl;
s and t are independently an integer from 1-5000.

In one embodiment in connection with the makeup composition, the weight of silicone gum in the makeup composition is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%.

In one embodiment in connection with the makeup composition, silicone resins are silicone material which is formed by branched, cage-like oligosiloxanes with the general formula RₐSiX_{b}O_{c}, wherein R is a C₁-C₆alkyl group, C₂₋₆alkenyl group, or a phenyl group; X is H, OH, Cl, or C₁₋₁₀alkoxy; a, b, or c is a number independently ranging from 0 to 4. These resins can be dissolved in a solvent, such as a polydimethylsiloxane or polyphenylsiloxane oil or a cyclomethicone. These resins can be modified by an ester group, or by an alkyl group. In one embodiment, the silicone resin is polymethylsilsequioxanes.

In one embodiment in connection with the makeup composition, silicone resins have a particle size ranging from about 0.01 μm to about 200 μm, about 0.1 μm to about 100 μm, about 0.5 μm to about 90 μm, about 1 μm to about 80 μm, about 2 μm to about 70 μm, about 3 μm to about 60 μm, about 4 μm to about 50 μm, about 5 μm to about 40 μm, about 5 μm to about 30 μm, about 5 μm to about 20 μm, about 5 μm to about 10 μm, or about 5 μm to about 8 μm.

In one embodiment in connection with the makeup composition, the weight of silicone resin in the makeup composition is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%.

In one embodiment in connection with the makeup composition, silicone compositions comprise silicone surfactants. The silicone surfactants comprise hydrophobic silicone and other functional groups as well. In some embodiments, a silicone surfactant is incorporated into a surface-active agent, with a polyoxyalkylene portion of the molecule and or a hydrocarbon portion of the molecule.

In one embodiment in connection with the makeup composition, the weight of silicone surfactants in the makeup composition can range from about 0.1% to about 50%, about 0.5% to about 50%, about 1% to about 50%, about 2% to about 50%, about 5% to about 50%, about 10% to about 50%, about 15% to about 50%, about 20% to about 50%, about 25% to about 50%, about 30% to about 50%, about 35% to about 50%, about 40% to about 50%, or about 45% to about 50%.

In one embodiment in connection with the makeup composition, the silicone is selected from dimethicone, methicone, trimethicone, cyclomethicone, amodimethicone, trimethylsilylamodimethicone, cyclotetrasiloxane, cyclopentasiloxane, cyclehexasiloxane, polydimethylsiloxane, polymethylsilsequioxane, dimethiconol, and mixtures thereof. In another embodiment, the silicone is polydimethylsiloxane.

In one embodiment in connection with the makeup composition, the makeup composition further comprises a coloring agent. In one embodiment, the coloring agent comprises pigments, nacres, and reflective particles, dyestuffs, and a mixture thereof.

It should be noted that the color, sunscreen, and gelling agents described in the method section above are also suitable to be applied in the makeup composition.

In another embodiment in connection with the makeup composition, the makeup composition may further comprise a moisturizer, emollient, humectant, thickener, skin toner, or a mixture thereof.

In another embodiment in connection with the makeup composition, the makeup composition is formulated such that, upon administration to a subject, it sufficiently conceals the scar to the extent that the scar is invisible to eyes while treating the scars simultaneously. The method to measure the extent of concealment is described in the method section above. In one embodiment, the makeup composition coverage ranges from about 30 to about 200, about 40 to about 200, about 50 to about 200, about 60 to about 200, about 70 to about 200, about 80 to about 200, about 90 to about 200, about 100 to about 200, about 100 to about 190, about 100 to about 180, about 100 to about 170, about 100 to about 160, about 100 to about 150, about 100 to about 140, about 100 to about 130, about 100 to about 120, or about 100 to about 110. Preferably, the coverage of the makeup compositions of the disclosure herein is about 30, about 50, about 80, about 100, about 150, or about 200.

In another embodiment, the makeup compositions contain less than 1%, or do not contain any, comedogenic compounds. Comedogenic compounds used herein refer to ingredients tending to clog pores of the skin. In one embodiment, the makeup compositions contain less than 1%, or do not contain any of the following compounds: sodium chloride, wheat germ oil, sodium lauryl sulfate, red algae, octyl stearate, laureth 4, isopropyl myristate, isopropyl isostearate, isocetyl stearate, carrageenan, algae extract, or combinations thereof.

In another embodiment, the makeup composition comprises silicone and color agents. In one embodiment, the ratio between silicone and color agents ranges from about 0.01 to about 99, about 0.1 to about 95, about 1 to about 90, about 5 to about 85, about 10 to about 80, about 15 to about 75, about 20 to about 70, about 25 to about 65, about 30 to about 60, about 35 to about 55, about 40 to about 50, or about 40 to about 45. In another embodiment, the ratio between silicones and color agents is about 0.01, about 0.05, about 0.1, about 0.5, about 1, about 5, about 10, about 15, about 20, about 25, about 30, about 35, about 40, about 45, about 50, about 55, about 60, about 65, about 70, about 75, about 80, about 85, about 90, about 95, or about 99.

### Scar Treatment Kit

In another aspect, the present disclosure provides a scar treatment kit comprising a) the makeup composition described herein; b) at least one container which contains the makeup composition; and c) at least one applicator. In one embodiment, the applicator in the kit is a brush, a puff, or a sponge.

In another embodiment in connection with the kit, the kit comprises a plurality of compartments for containing an amount of makeup composition arranged within rows and columns. In one embodiment, the compartments of the kit are arranged based on the color of the makeup composition. In another embodiment, the shape of the compartments is a circle, triangle, rectangle, oval, square, diamond, trapezoid, or pentagon.

### EXAMPLES

The following examples are provided to promote a more clear understanding of certain embodiments of the present invention. It should be understood that the Examples are given by way of illustration only. From the above discussion and the Examples, one skilled in the art can ascertain the essential characteristics of the invention, and without departing from the spirit and scope thereof, can make various changes and modifications to adapt the invention to various uses and conditions. As a result, the invention is not limited by the illustrative examples set forth hereinbelow, but rather is defined by the claims appended hereto.

### Example 1

a. Preparation of Makeup Composition I

65 g polymethylsilsesquioxane, 0.1 g cetyl dimethicone, 1.6 g behenoxy dimethicone, 0.5 g trimethylsiloxysilicate, and 2.8 g polydimethyl siloxane are mixed together. The mixture is ground and sifted to afford part A. Then 10 g titanium dioxide, 0.5 g D&C red 30, 10.0 g mica-titanium, 4.0 g zinc stearate, and 5.5 g talc are mixed, ground and sifted to afford part B. Part A and part B are mixed together and ground until a homogeneous powder is formed. The powder is further sifted to afford makeup composition I which is packed in a metal container.

b. Coverage Test

Makeup composition I is drawn down on a polyester film with a control mil thickness. The polyester is put upon a LENETA card. Reflectance, Ro (black background) and Rw (white background), is measured by using a spectrophotometer. Each LENETA card can read 3 data points on the black background and this can be repeated for 6 different LENEAT cards. The 18 data points are collected and the contrast ratio is determined based on the formula C=100*Ro/Rw.

c. SPF Value

Makeup Composition I is tested on 5 subjects for Sun protection (SPF) according to the methods described in Federal Register Vol. 46, No. 17, of Tuesday Jan. 27, 1981. The SPF results are expected to be about 20.

d. Method of Use

Makeup composition I (1 g) is rubbed onto a keloid scar on the skin with a soft massage once in the morning and once in the afternoon. The soft massage ensures intimate contact with the skin. Upon the application of makeup composition I, the keloid scar is invisible to the eye and concealed.

### Example 2

a. Rabbit Ear Model

Using a dermal biopsy punch and a dissecting microscope, 7-mm wounds are made on the ventral surface of each ear of four 3-kg New Zealand White rabbits (6 wounds on each ear). The wounds involve complete removal of the epithelium, dermis, and perichondrium. The wounds are covered with a polyurethane film until healing is complete (17 days). The wounds on one ear are then treated once daily for 30 days by topical application of makeup composition I of Example 1. The six wounds on the other ear serve as untreated controls. The animals are sacrificed on day 36 of the experiment.

Scar tissue is harvested, bisected at the highest point of the scar, and processed for histological analysis using hematoxylin and eosin staining. An observer blinded to the treatment quantifies scar elevation by measuring the “scar elevation index” under 100× magnification. The scar elevation index is calculated as the area of the scar dermis (S) divided by the normal area of dermis (N), the latter being based on the height of the dermis in adjacent, unwounded skin (R. Reid et al., J Plast Reconstr Aesthet Surg. (2007), 60:64-72).

The mean scar elevation index in the control animals (N=20) is expected to be about 1.7, and the mean scar elevation index in the treated animals (N=19) is expected to be about 1.40.

### Example 3

Table 1 provides formulations of nine different embodiments.

TABLE 1

| | comp. * | comp. | comp. | comp. | comp. | comp. | comp. | comp. | comp. |
|---|---|---|---|---|---|---|---|---|---|
| | 2 | 3 | 4 | 5 | 6 | 7 | 8 | 9 | 10 |
| silicone | 50% | 75% | — | — | — | — | 25% | — | — |
| oil | | | | | | | | | |
| silicone | 20% | — | 75% | — | — | — | — | 25% | — |
| wax | | | | | | | | | |
| silicone | 15% | — | — | 70% | — | — | — | — | 25% |
| gum | | | | | | | | | |
| silicone | 2% | — | — | — | 75% | 25% | — | — | — |
| resin | | | | | | | | | |
| silicone | 1% | 5% | 5% | 5% | 5% | 2% | 5% | 3% | 10% |
| surfac- | | | | | | | | | |
| tant | | | | | | | | | |
| coloring | 5% | 5% | 3% | 5% | 5% | 10% | 15% | 10% | 25% |
| agent | | | | | | | | | |
| sun- | 2% | 10% | 15% | 15% | 18% | 60% | 55% | 60% | 40% |
| screen | | | | | | | | | |
| agent | | | | | | | | | |
| gelling | 7% | 5% | 2% | 5% | 2% | 3% | — | 2% | — |
| agent | | | | | | | | | |

* comp. means makeup composition.

### Example 4

Table 2 provides formulations of nine different embodiments

TABLE 2

| | comp. * | comp. | comp. | comp. | comp. | comp. | comp. | comp. | comp. |
|---|---|---|---|---|---|---|---|---|---|
| | 11 | 12 | 13 | 14 | 15 | 16 | 17 | 18 | 19 |
| silicone | 40-60% | 70-90% | — | — | — | — | 20-30% | — | — |
| oil | | | | | | | | | |
| silicone | 15-25% | — | 70-90% | — | — | — | — | 20-30% | — |
| wax | | | | | | | | | |
| silicone | 10-20% | — | — | 60-90% | — | — | — | — | 20-30% |
| gum | | | | | | | | | |
| silicone | 1-5% | — | — | — | 70-90% | 20-30% | — | — | — |
| resin | | | | | | | | | |
| silicone | 0.5-5% | 0.5-5% | 3-7% | 3-7% | 3-7% | 0-5% | 3-7% | 0-5% | 5-15% |
| surfactant | | | | | | | | | |
| coloring | 3-7% | 3-7% | 1-5% | 3-7% | 3-7% | 5-15% | 10-20% | 5-15% | 20-30% |
| agent | | | | | | | | | |
| sunscreen | 0-5% | 1-10% | 10-20% | 10-20% | 10-20% | 50-70% | 40-60% | 50-70% | 30-50% |
| agent | | | | | | | | | |
| gelling | 5-10% | 3-7% | 1-5% | 3-7% | 1-5% | 1-5% | — | 1-5% | — |
| agent | | | | | | | | | |

* comp. means makeup composition.

### Example 5

Table 3 provides five formulations of makeup compositions containing cortisone

TABLE 3

| | comp.* 20 | comp. 21 | comp. 22 | comp. 23 | comp. 24 |
|---|---|---|---|---|---|
| silicone oil | 40-60% | 70-90% | — | — | — |
| silicone wax | 15-25% | — | 70-90% | — | — |
| silicone gum | 10-20% | — | — | 60-90% | — |
| silicone resin | 1-5% | — | — | — | 70-90% |
| silicone | 0.5-5% | 0.5-5% | 3-7% | 3-7% | 3-7% |
| surfactant | | | | | |
| coloring | 3-7% | 3-7% | 1-5% | 3-7% | 3-7% |
| agent | | | | | |
| sunscreen | 0-5% | 1-10% | 10-20% | 10-20% | 10-20% |
| agent | | | | | |
| gelling agent | 5-10% | 3-7% | 1-5% | 3-7% | 1-5% |
| Cortisone | 0-2% | 0.5-1% | 0-1% | 0.5-2% | 0-0.5% |

*comp. means makeup composition.

### Example 6

Table 4 provides five formulations of makeup compositions containing cortisone and another scar melting agent.

TABLE 4

| | comp.* 25 | comp. 26 | comp. 27 | comp. 28 | comp. 29 |
|---|---|---|---|---|---|
| silicone oil | 40-60% | 70-90% | — | — | — |
| silicone wax | 15-25% | — | 60-80% | — | — |
| silicone gum | 10-20% | — | — | 50-70% | — |
| silicone resin | 1-5% | — | — | — | 70-85% |
| silicone | 0.5-5% | 0.5-5% | 3-7% | 3-7% | 3-7% |
| surfactant | | | | | |
| coloring | 3-7% | 3-7% | 1-5% | 3-7% | 3-7% |
| agent | | | | | |
| sunscreen | 0-5% | 1-10% | 10-20% | 10-20% | 10-20% |
| agent | | | | | |
| gelling agent | 5-10% | 3-7% | 1-5% | 3-7% | 1-5% |
| cortisone | 0-2% | 0.5-1% | 0-1% | 0.5-2% | 0-0.5% |
| collagenase | 0-0.5% | — | — | — | — |
| protease | — | 0-0.5% | — | — | — |
| oligopoly- | — | — | 0-10% | 10-20% | — |
| saccharide | | | | | |
| biologics | — | — | — | — | 0-5% |

*comp. means makeup composition.

What is claimed is:

1. A method of treating and concealing a skin defect in a subject comprising applying a makeup composition to the skin defect, wherein the makeup composition comprises at least 20% by weight silicone and a coloring agent, wherein the makeup composition simultaneously conceals and treats said skin defect.

2. The method of claim 1, wherein the skin defect is a scar.

3. The method of claim 2, wherein the scar is selected from a keloid scar, a hypertrophic scar, a contracture scar, an acne scar, and a surgical scar.

4. The method of claim 2, wherein the scar is a surgical scar following suture removal beginning about 7 to about 14 days after surgery.

5. The method of claim 1, wherein the makeup composition further provides UV protection.

6. The method of claim 1, wherein the skin is substantially healed after the treatment and a scar is forming.

7. The method of claim 2, wherein the treatment results in substantial reduction in the amount and visibility of scar tissue.

8. The method of claim 2, wherein the size of the scar is reduced after the treatment.

9. The method of claim 7, wherein the treatment further provides a calming effect to the skin.

10. The method of claim 1, wherein the makeup composition contains a nacreous pigment.

11. The method of claim 10, wherein the nacreous pigment is titanium mica with iron oxides.

12. The method of claim 1, wherein the silicone is selected from liquid silicone, oil silicone, wax silicone, resin silicone, or mixtures thereof.

13. The method of claim 1, wherein the silicone is selected from dimethicone, methicone, trimethicone, cyclomethicone, amodimethicone, trimethylsilylamodimethicone, cyclotetrasiloxane, cyclopentasiloxane, cyclehexasiloxane, polydimethylsiloxane, polymethylsilsequioxane, and dimethiconol, and mixtures thereof.

14. The method of claim 1, wherein the volume of the makeup composition applied to the skin defect is about 0.1 to about 10 mL.

15. The method of claim 1, wherein the viscosity of the silicone is about 1000 cP to about 1,000,000 cP.

16. The method of claim 1, wherein the makeup composition is maintained on the skin for at least 1-8 hours daily.

17. The method of claim 16, wherein the makeup composition is applied 1-5 times daily for about 14 days to about 120 days.

18. The method of claim 1, wherein the weight of silicone is at least 25% with respect to the total weight of the makeup composition.

19. The method of claim 18, wherein the weight of silicone is at least 30% with respect to the total weight of the makeup composition.

20. The method of claim 1, wherein the subject is a human.

21. The method of claim 1, wherein the makeup composition further comprises cortisone.

22. A makeup composition comprising at least 80% by weight of silicone and a coloring agent, wherein the composition, when applied to a scar on the skin of a subject, treats said scar on the skin of the subject while simultaneously camouflaging the scar and providing UV protection.

23. The makeup composition of claim 22, wherein the silicone comprises polydimethylsiloxane.

24. The makeup composition of claim 22, wherein the composition further comprises cortisone.

25. A method of treating and concealing a skin defect in a subject comprising applying a makeup composition to the skin defect, wherein the makeup composition comprises at least 20% by weight silicone, a coloring agent, and a scar melting agent selected from the group consisting of steroid hormones, collagenases, proteases, oligopolysaccharides, and biologics, and combinations thereof, wherein said makeup composition simultaneously conceals and treats said skin defect.

26. The method of claim 25, wherein the steroid hormone is cortisone.

27. A method of simultaneously treating and concealing a skin defect in a subject comprising applying a makeup composition to the skin defect, wherein the makeup composition comprises at least 20% by weight silicone and a coloring agent; wherein the coloring agent deposits color onto a keratinous material matching that of the keratinous material; and wherein the makeup composition is not comprised of immiscible layers.

28. The method of claim 27, wherein the skin defect is a scar.

29. The method of claim 27, wherein the silicone is selected from liquid silicone, oil silicone, wax silicone, resin silicone, or mixtures thereof.

30. The method of claim 27, wherein the subject is a human.
